(19) United States
(12) Patent Application Publication
(10) Pub. No.: US 2014/0193375 A1
(43) Pub. Date: Jul. 10, 2014

(54) Mesenchymal Stromal Cell Populations and Methods of Making Same

(71) Applicants: Frank Zeigler, Encinitas, CA (US); Ronnda L. Bartel, San Diego, CA (US)

(72) Inventors: Frank Zeigler, Encinitas, CA (US); Ronnda L. Bartel, San Diego, CA (US)

(73) Assignee: AASTROM BIOSCIENCES, INC., Ann Arbor, MI (US)

(21) Appl. No.: 14/117,774

(22) PCT Filed: May 18, 2012
(86) PCT No.: PCT/US12/38602

§ 371 Date: Feb. 14, 2014

Related U.S. Application Data
(60) Provisional application No. 61/487,558, filed on May 18, 2011.

(51) Int. Cl. A61K 35/28

(52) U.S. Cl. CPC A61K 35/28
USPC 424/93.7; 435/325

(57) ABSTRACT

The present invention provides compositions of mesenchymal stromal cells which express B7-H3, their subsequent use in tissue repair, improved methods of producing tissue repair cells and method of producing a substantially pure population of CD14+ autofluorescent macrophages.

### RELATED APPLICATIONS

This patent application claims priority to U.S. Provisional Application No. 61/487,558, filed May 18, 2011, the contents of which is incorporated herein by reference in its entirety.

### FIELD OF THE INVENTION

The present invention relates to compositions of mesenchymal stromal cells, their subsequent use in tissue repair, improved methods of producing tissue repair cells and method of producing a substantially pure population of CD14⁺ autofluorescent macrophages.

### BACKGROUND OF THE INVENTION

Regenerative medicine harnesses, in a clinically targeted manner, the ability of regenerative cells, e.g., stem cells and/or progenitor cells (i.e., the unspecialized master cells of the body), to renew themselves indefinitely and develop into mature specialized cells.

Stem cells are found in embryos during early stages of development, in fetal tissue and in some adult organs and tissue. Embryonic stem cells (hereinafter referred to as “ESCs”) are known to become many if not all of the cell and tissue types of the body. ESCs not only contain all the genetic information of the individual but also contain the nascent capacity to become any of the 200+ cells and tissues of the body. Thus, these cells have tremendous potential for regenerative medicine. For example, ESCs can be grown into specific tissues such as heart, lung or kidney which could then be used to repair damaged and diseased organs. However, ESC derived tissues have clinical limitations. Since ESCs are necessarily derived from another individual, i.e., an embryo, there is a risk that the recipient's immune system will reject the new biological material. Although immunosuppressive drugs to prevent such rejection are available, such drugs are also known to block desirable immune responses such as those against bacterial infections and viruses.

Moreover, the ethical debate over the source of ESCs, i.e., embryos, is well-chronicled and presents an additional and, perhaps, insurmountable obstacle for the foreseeable future.

Adult stem cells (hereinafter interchangeably referred to as “ASCs”) represent an alternative to the use of ESCs. ASCs reside quietly in many non-embryonic tissues, presumably waiting to respond to trauma or other destructive disease processes so that they can heal the injured tissue. Notably, emerging scientific evidence indicates that each individual carries a pool of ASCs that may share with ESCs the ability to become many if not all types of cells and tissues. Thus, ASCs, like ESCs, have tremendous potential for clinical applications of regenerative medicine.

ASC populations have been shown to be present in one or more of bone marrow, skin, muscle, liver and brain. However, the frequency of ASCs in these tissues is low. For example, mesenchymal stem cell frequency in bone marrow is estimated at between 1 in 100,000 and 1 in 1,000,000 nucleated cells Thus, any proposed clinical application of ASCs from such tissues requires increasing cell number, purity, and maturity by processes of cell purification and cell culture.

Although cell culture steps may provide increased cell number, purity, and maturity, they do so at a cost. This cost can include one or more of the following technical difficulties: loss of cell function due to cell aging, loss of potentially useful cell populations, delays in potential application of cells to patients, increased monetary cost, increased risk of contamination of cells with environmental microorganisms during culture, and the need for further post-culture processing to deplete culture materials contained with the harvested cells.

More specifically, all final cell products must conform with rigid requirements imposed by the Federal Drug Administration (FDA). The FDA requires that all final cell products must minimize “extraneous” proteins known to be capable of producing allergenic effects in human subjects as well as minimize contamination risks. Moreover, the FDA expects a minimum cell viability of 70%, and any process should consistently exceed this minimum requirement.

While there are existing methods and apparatus for separating cells from unwanted dissolved culture components and a variety of apparatus currently in clinical use, such methods and apparatus suffers from a significant problem—cellular damage caused by mechanical forces applied during the separation process, exhibited, for instance, by a reduction in viability and biological function of the cells and an increase in free cellular DNA and debris. Furthermore, significant loss of cells can occur due to the inability to both transfer all the cells into the separation apparatus as well as extract all the cells from the apparatus. In addition, for mixed cell populations, these methods and apparatus can cause a shift in cell profile due to the preferential loss of larger, more fragile subpopulations.

Thus, there is a need in the field of cell therapy, such as tissue repair, tissue regeneration, and tissue engineering, for cell compositions that are ready for direct patient administration with substantially high viability and functionality, and with substantial depletion of materials that were required for culture and harvest of the cells. Furthermore, there are needs for reliable processes and devices to enable production of these compositions that are suitable for clinical implementation and large-scale commercialization of these compositions as cell therapy products

### SUMMARY OF THE INVENTION

The present invention provides an isolated mesenchymal stromal cell composition where the mesenchymal stromal cell expresses B7 homolog 3(B7-H3). Also provided herein is an isolated cell composition that comprises mesenchymal stromal cells expressing B7-H3. The present invention further provides a mesenchymal stromal cell that has been genetically engineered to stably express B7-H3 on the surface of the cell.

Any cell composition of the present invention may comprise mesenchymal stromal cells that are non-proliferative after less than 5 passages in the culture. Alternatively, any cell composition of the present invention may comprise mesenchymal stromal cells that do not differentiate in culture. Optionally, the mesenchymal stromal cells may have been immortalized. Preferably, the cell composition of the present invention comprises at least 70%, 80% or 90% of the cells expressing CD90. In a preferred embodiment, the cell composition of the present invention comprises less than 25% viable CD45⁺ cells. The mesenchymal stromal cells of the present invention may be adherent in culture.

Any mesenchymal stromal cells of the cell composition can be derived from hematopoietic cells or mononuclear cells. The mononuclear cells are derived from mobilized peripheral blood, bone marrow, umbilical cord blood or fetal liver.

The cell composition of the present invention can inhibit T-cell activation or promote the expansion of Th2 type CD8 (Th2/CD8) lymphocytes.

The cell composition of the present invention may comprise mesenchymal stromal cells that are CD90⁺ and CD105⁺. Preferably, the cell composition of the present invention may comprise mesenchymal stromal cells that are CD90⁺, CD105⁺, CD146⁺ and CD73⁺. In another preferred embodiment, any cell composition of the invention contains less than 2 μg/ml of bovine serum albumin; less than 1 μg/ml of a enzymatically active harvest reagent; and substantially free of mycoplasm, endotoxin, and microbial contamination.

The present invention also provides a method of tissue regeneration or repair by administering to a patient in need of any cell composition of the invention. The tissue is selected from the group consisting of cardiac tissue, bone tissue, neuronal tissue, skin tissue, lung tissue, salivary gland tissue, liver tissue, and pancreatic tissue.

Another aspect of the invention is a method of producing a cell composition that comprises a mixed population of cells of hematopoietic, mesenchymal and endothelial lineage and is characterized as containing 5-75% viable CD90^{|} cells with the remaining cells in said composition being CD45⁻, CD31⁺, CD14⁻, and auto⁺, by culturing mononuclear cells in the presence of a B7-H3 polypeptide and or a V-set and Ig domain-containing 4 (VSIG4) polypeptide.

A further aspect of the invention provides a method of producing a substantially pure population of CD14⁺ autofluorescent macrophages by culturing mononuclear cells in the presence of a B7-H3 polypeptide and or a V-set and Ig domain-containing 4 (VSIG4) polypeptide and isolating said CD14⁺ autofluorescent macrophages from the culture.

Also included in the invention is method of producing a substantially pure population of CD14⁺ autofluorescent macrophages by culturing mononuclear cells in the presence of any cell composition of the invention and isolating said CD14⁺ autofluorescent macrophages from the culture.

In any of the methods, the macrophages express at least one of the following markers: CD45, CD163 or CD206. The culturing is performed by providing a biochamber for culturing cells; providing a culture media for culturing cells within biochamber; inoculating the biochamber with cells; and culturing the cells. The culturing may further comprises upon a predetermined time period of culture, displacing the culture media from the biochamber with a biocompatible first rinse solution; replacing the first rinse solution with a cell harvest enzyme solution; incubating the contents of the biochamber for a predetermined period of time, wherein during incubation, the enzyme at least dissociates the cells from each other and/or from the biochamber surface; displacing the enzyme solution with a second rinse solution, wherein upon the enzyme being displaced in the chamber is substantially filled with the second rinse solution; displacing a portion of the second rinse solution with a gas to obtain a predetermined reduced liquid volume in the chamber; agitating the chamber to bring settled cells into suspension; and draining the solution with suspended cells into a cell collection container.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 shows microarray gene expression analysis of TRCs (IXMYELOCEL-T).

FIG. 2 shows FACS analysis of TRCs (IXMYELOCEL-T) demonstrating B7H3 expression of CD90⁻ MSCs and VSIG4 expression on CD14⁺ macrophages.

FIG. 3 shows that B7H3 neutralizing antibody alters TH1/TH2 lymphocyte differentiation.

FIG. 4 shows reduced response to allogeneic T-cell proliferation.

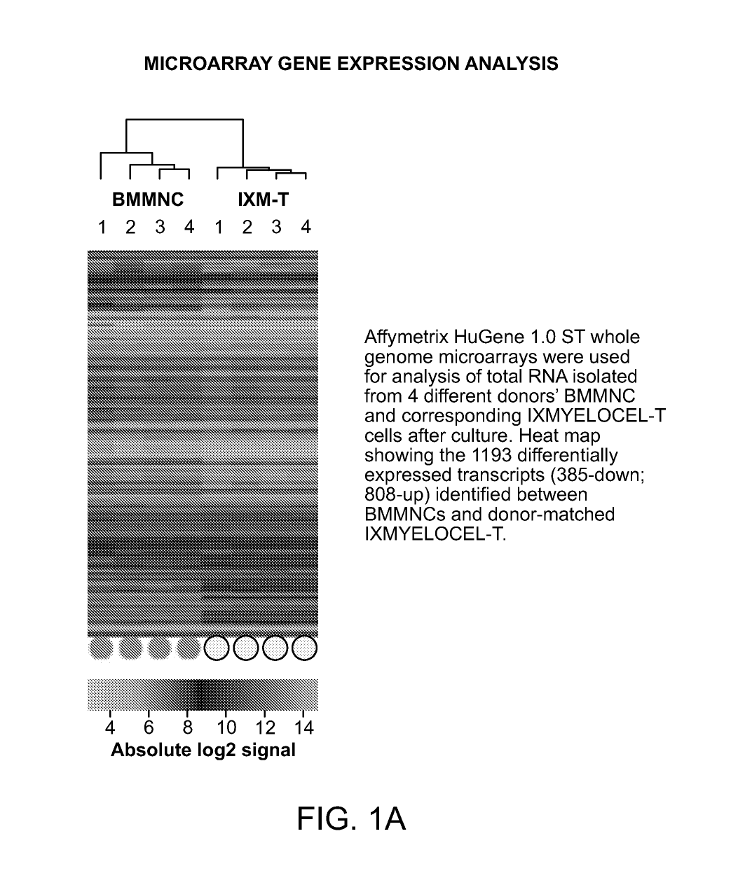

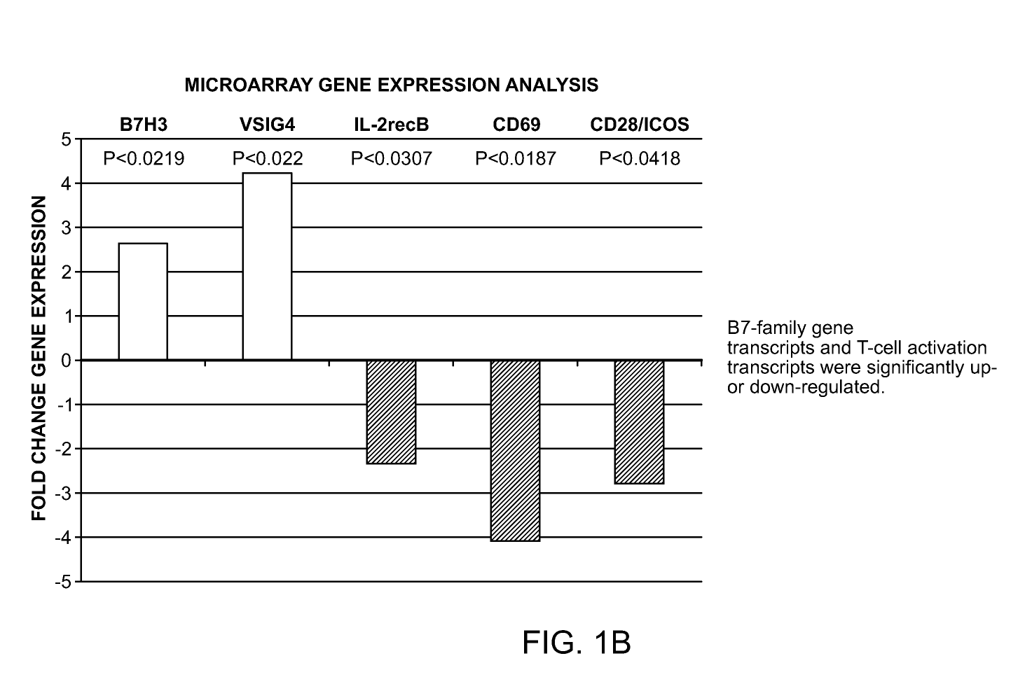

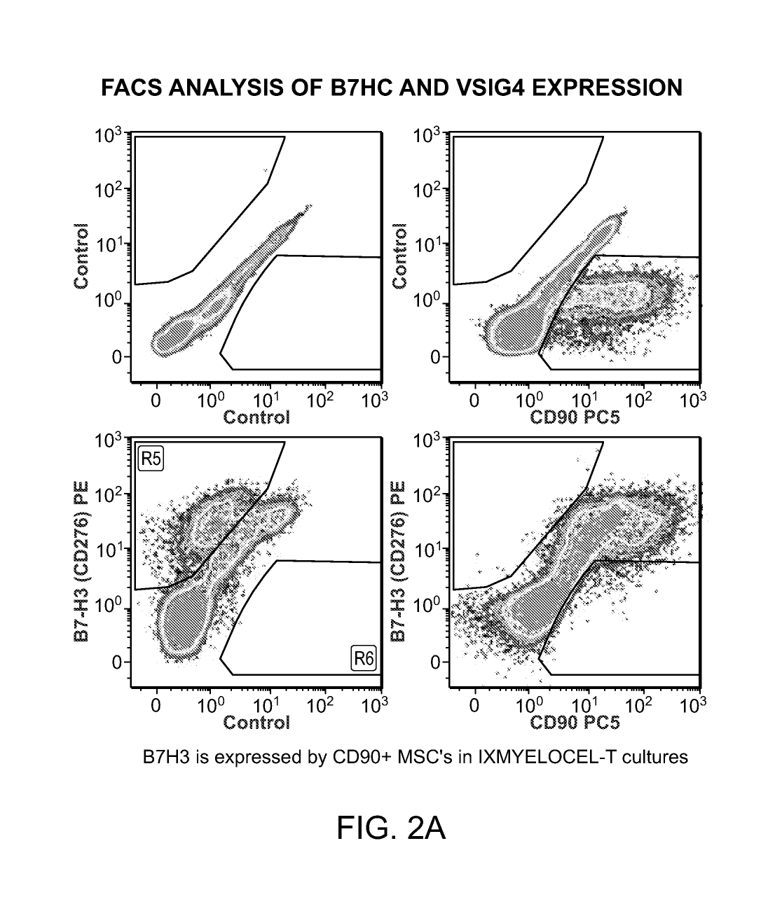

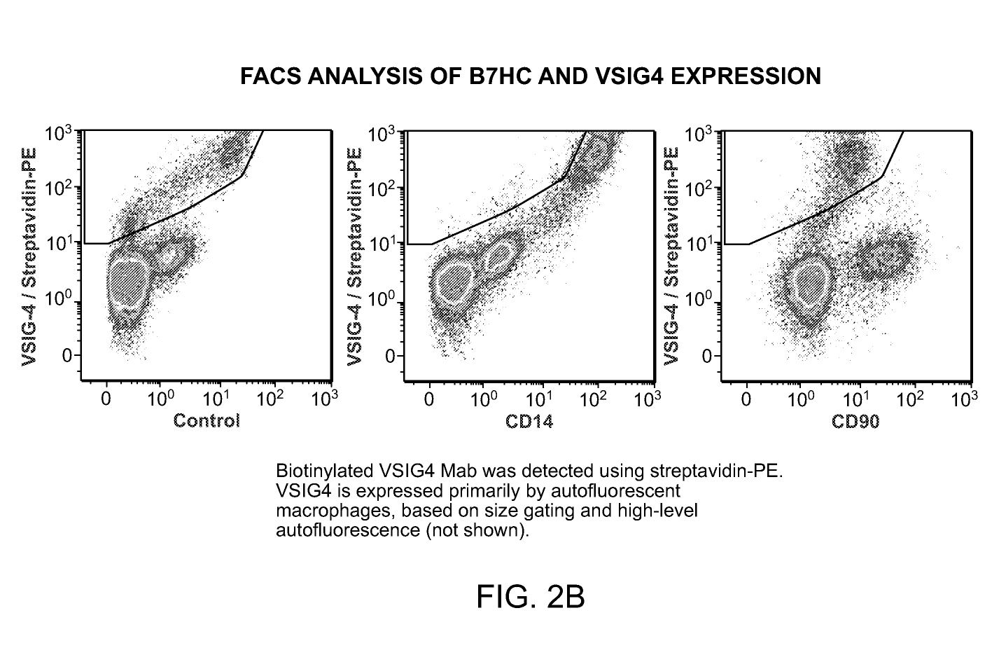

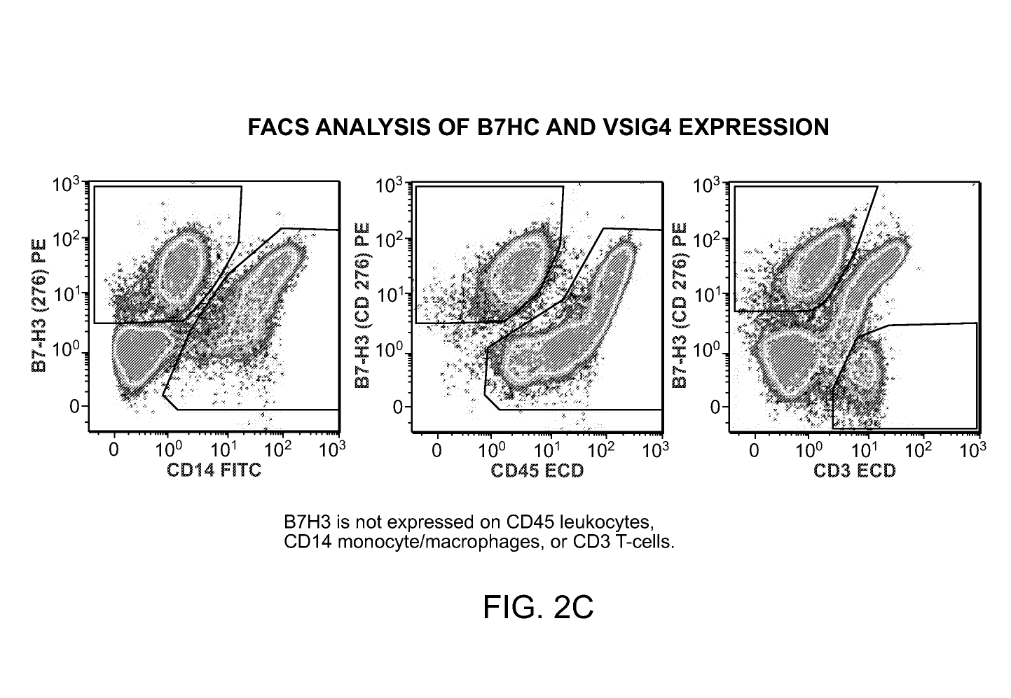

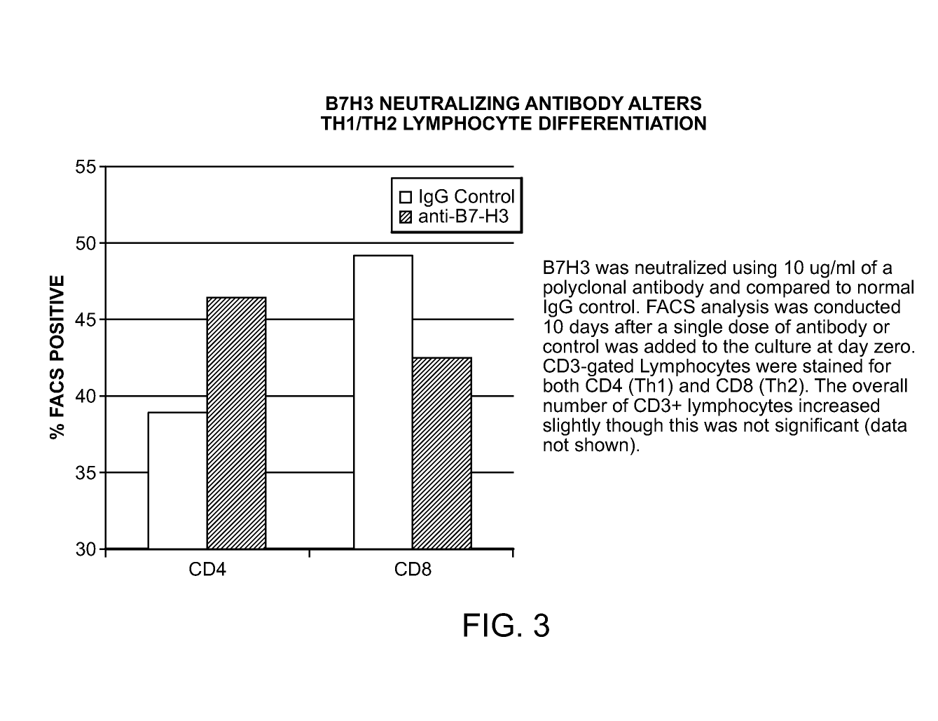

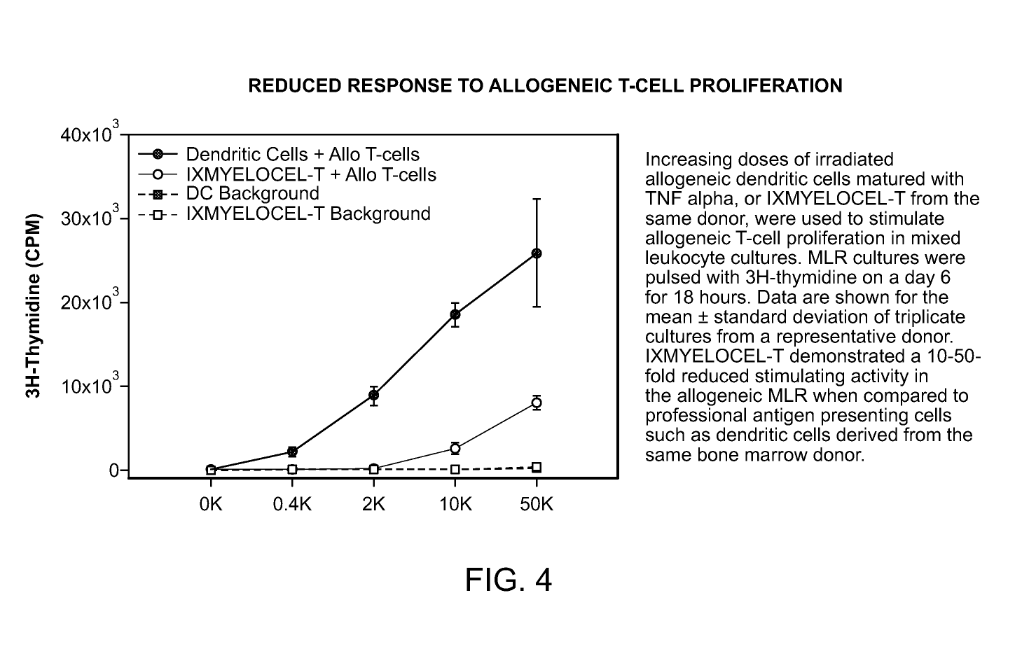

### DETAILED DESCRIPTION OF THE INVENTION

The present invention is based on the surprising discovery that a mixed population of cells that are enhanced in stem and progenitor cells (referred to herein as “Tissue Repair Cells” or “TRCs”) contain mesenchymal stromal cells expressing B7 homolog 3 (B7-H3). The cells modulate the immune response in vitro. This is the first report constitutive expression of B7H3 on autologous mesenchymal stromal cells. The mesenchymal stromal cells express CD90^{|} and are derived from cells of hematopoietic lineage. Surprisingly, the mesenchymal stromal cells of the invention, in contrast to mesenchymal stem cells, do not substantially expand in culture and do no differentiate.

Tissue Repair Cells (TRCs) are an autologous, bone marrow derived, mixed cell product composed of hematopoietic cell types and mesenchymal stromal cells, which has shown clinical efficacy in ischemic tissue repair. Gene expression studies using microarrays were conducted which compared the global gene expression profiles of bone marrow mononuclear cells (BMMNCs) from 4 different donors, to their matched, autologous TRC products after culture. The data demonstrated that a number of transcripts involved in T-cell activation were significantly down-regulated, including IL-2 receptor (2.33-fold, p<0.00025), ICOS/CTLA-4 (2.79-fold; p<0.00094), and CD69 (4.08-fold; p<0.000018). Concomitantly, these studies identified 2 members of the B7 superfamily which were significantly up-regulated during the expansion of autologous mesenchymal stromal cells in the TRC manufacturing process, B7H3 (2.65-fold; p<0.000091) and VSIG4 (4.23-fold; p<0.00042). These results are confirmed by phenotypic analysis using FACS and antibodies for both VSIG4 and B7H3. VSIG4, which is a negative regulator of T-cell activation, was co-expressed by CD14⁻ macrophages. However, B7H3 was exclusively co-expressed by CD90^{|}/CD105^{|} mesenchymal stromal cells, and not on CD45^{|} hematopoietic cells. Furthermore, we evaluated the functional significance of autologous mesenchymal stromal cells which express B7H3, in allogeneic mixed lymphocyte reactions, and found that TRCs inhibit T-cell activation in the allogeneic mixed lymphocyte response, even after stimulation with gamma-interferon or anti-CD3 antibody. These results demonstrate that TRCs are inhibitory to T-cell activation in vitro.

Accordingly, the invention provides an isolated mesenchymal stromal cell composition where the mesenchymal stromal cells expresses B7 homolog 3 (B7-H3 or B7H3). Also included in the invention are cell compositions containing mesenchymal stromal cells expressing B7 homolog 3 (B7-H3). In some aspects, these compositions contain at least 70%, 80%, or 90% cells expressing CD90. In some aspects, these compositions contain less than 25%, 20%, 10%, or 5% CD45⁺ cells. The mesenchymal stromal cell compositions do not substantially expand in culture . For example, the mesenchymal stromal cell compositions are non-proliferative (i.e. do not undergo cell division) after less than 5 passages in culture. In some aspect the mesenchymal stromal cell compositions are non-proliferative after 4, 3, 2 or 1 passage in culture. The mesenchymal stromal cells are CD90⁺ and CD105⁺. Optionally, the mesenchymal stromal cells are CD146⁺ and CD73⁺. Alternatively, the mesenchymal stromal cells are CD90, CD105^{|}. CD146^{|} and CD73^{|}. The cells are derived from cells of hematopoietic lineage. Preferably, the cells are derived from mononuclear cells. The mononuclear cells are derived from mobilized peripheral blood, bone marrow, umbilical cord blood or fetal liver. The composition inhibits T cell activation, alters lymphocyte differentiation and enhances (i.e., promotes expansion of) Th2-type CD8 (Th2/CD8) cells.

The cell compositions are useful for tissue regeneration or repair by administering to a patient in need thereof the mesenchymal stromal cell compositions of the invention. The tissue is for example selected from cardiac tissue, bone tissue, neuronal tissue, skin tissue, lung tissue, salivary gland tissue, liver tissue, or pancreatic tissue.

The mesenchymal stromal cell of the invention may be isolated from a TRC composition by positive selection using B7-H3 and optionally CD90 and CD105. Methods of positive selection are known in the art.

The mesenchymal stromal cell of the invention may also be immortalized such that they may be expanded in vivo. Methods of immortalizing cells are known in the art.

Also included in the invention is a mesenchymal stromal cell, i.e., a CD90⁺ cell that has been genetically engineered to stably express a B7-H3 polypeptide on the surface of the cell. Optionally, the cell is further immortalized such that it can be expanded in culture.

Also included in the invention is an improved process for culturing TRCs, which includes culturing mononuclear cells in the presence of a B7-H3 polypeptide, a VSIG4 or both. Further included is a substantially purified population of CD14⁺ autofluorescent (auto⁺) macrophages. The substantially purified population of CD14⁺ autofluorescent macrophages is produced using the improved process of culturing TRCs and isolating CD14⁻ autofluorescent macrophages.

Isolation, purification, characterization, and culture of TRCs is described in WO/2008/054825, the contents of which are incorporated by reference its entirety.

This invention also provides a method of producing a cell composition that comprises a mixed population of cells of hematopoietic, mesenchymal and endothelial lineage and is characterized as containing 5-75% viable CD90⁺ cells with the remaining cells in said composition being CD45⁺, CD31⁺, CD14⁺, and auto⁺, by culturing mononuclear cells in the presence of a B7-H3 polypeptide and or a V-set and Ig domain-containing 4 (VSIG4) polypeptide.

Further provided is a method of producing a substantially pure population of CD14⁺ autofluorescent macrophages by culturing mononuclear cells in the presence of a B7-H3 polypeptide and or a V-set and Ig domain-containing 4 (VSIG4) polypeptide and isolating said CD14^{|} autofluorescent macrophages from the culture.

Also included in the invention is method of producing a substantially pure population of CD14⁺ autofluorescent macrophages by culturing mononuclear cells in the presence of any cell composition of the invention and isolating said CD14⁺ autofluorescent macrophages from the culture.

In any of the methods, the macrophages express at least one of the following markers: CD45, CD163 or CD206. The culturing is performed by providing a biochamber for culturing cells; providing a culture media for culturing cells within biochamber; inoculating the biochamber with cells; and culturing the cells. The culturing may further comprises upon a predetermined time period of culture, displacing the culture media from the biochamber with a biocompatible first rinse solution; replacing the first rinse solution with a cell harvest enzyme solution; incubating the contents of the biochamber for a predetermined period of time, wherein during incubation, the enzyme at least dissociates the cells from each other and/or from the biochamber surface; displacing the enzyme solution with a second rinse solution, wherein upon the enzyme being displaced in the chamber is substantially filled with the second rinse solution; displacing a portion of the second rinse solution with a gas to obtain a predetermined reduced liquid volume in the chamber; agitating the chamber to bring settled cells into suspension; and draining the solution with suspended cells into a cell collection container.

MSCs expressing B7-H3 are useful for a variety of therapeutic methods including, tissue repair, tissue regeneration, and tissue engineering. For example, the TRC are useful in bone regeneration, cardiac regeneration, vascular regeneration, neural regeneration and the treatment of ischemic disorders. Ischemic conditions include, but are not limited to, limb ischemia, congestive heart failure, cardiac ischemia, kidney ischemia and ESRD, stroke, and ischemia of the eye. Additionally, because of the immuno-regulatory cytokines produced by the MSCs, the MSCs are also useful in the treatment of a variety of immune and inflammatory diseases. Immune and inflammatory diseases include for example, diabetes (Type I and Type II), inflammatory bowel diseases (IBD), graft verses host disease (GVHD), psoriasis, rejection of allogeneic cells, tissues or organs (tolerance induction), heart disease, spinal cord injury, rheumatoid arthritis, osteo-arthritis, inflammation due to hip replacement or revision, Crohn's disease, autoimmune diseases such as system lupus erythematosus (SLE), rheumatoid arthritis (RA), and multiple sclerosis (MS). In another aspect of the invention mesenchymal stromal cells are also useful for inducing angiogenesis.

Mesenchymal stromal cells are administered to mammalian subjects, e.g., human, to effect tissue repair or regeneration. The mesenchymal stromal cells are administered allogeneically or autogeneically.

The described mesenchymal stromal cells can be administered as a pharmaceutically or physiologically acceptable preparation or composition containing a physiologically acceptable carrier, excipient, or diluent, and administered to the tissues of the recipient organism of interest, including humans and non-human animals. Mesenchymal stromal cell-containing composition can be prepared by resuspending the cells in a suitable liquid or solution such as sterile physiological saline or other physiologically acceptable injectable aqueous liquids. The amounts of the components to be used in such compositions can be routinely determined by those having skill in the art.

The mesenchymal stromal cells or compositions thereof can be administered by placement of the TMSC suspensions onto absorbent or adherent material, i.e., a collagen sponge matrix, and insertion of the TRC-containing material into or onto the site of interest. Alternatively, the mesenchymal stromal cells can be administered by parenteral routes of injection, including subcutaneous, intravenous, intramuscular, and intrasternal. Other modes of administration include, but are not limited to, epicardial, endocardial intranasal, intrathecal, intracutaneous, percutaneous, enteral, and sublingual. In one embodiment of the present invention, administration of the mesenchymal stromal cells can be mediated by endoscopic surgery, such as thoracoscopy.

For injectable administration, the composition is in sterile solution or suspension or can be resuspended in pharmaceutically- and physiologically-acceptable aqueous or oleaginous vehicles, which may contain preservatives, stabilizers, and material for rendering the solution or suspension isotonic with body fluids (i.e. blood) of the recipient. Non-limiting examples of excipients suitable for use include water, phosphate buffered saline, pH 7.4, 0.15 M aqueous sodium chloride solution, dextrose, glycerol, dilute ethanol, and the like, and mixtures thereof. Illustrative stabilizers are polyethylene glycol, proteins, saccharides, amino acids, inorganic acids, and organic acids, which may be used either on their own or as admixtures. The amounts or quantities, as well as the routes of administration used, are determined on an individual basis, and correspond to the amounts used in similar types of applications or indications known to those of skill in the art.

Consistent with the present invention, the mesenchymal stromal cells can be administered to body tissues, including liver, pancreas, lung, salivary gland, blood vessel, bone, skin, cartilage, tendon, ligament, brain, hair, kidney, muscle, cardiac muscle, nerve, skeletal muscle, joints, and limb.

The number of cells in a mesenchymal stromal cells suspension and the mode of administration may vary depending on the site and condition being treated.

### Tissue Repair Cells (TRCs)

TRCs contain a mixture of cells of hematopoietic, mesenchymal and endothelial cell lineage produced from mononuclear cells. The mononuclear cells are isolated from adult, juvenile, fetal or embryonic tissues. For example, the mononuclear cells are derived from mobilized peripheral blood, bone marrow, peripheral blood, umbilical cord blood or fetal liver tissue. TRCs are produced from mononuclear cells, for example by an in vitro culture process which results in a unique cell composition having both phenotypic and functional differences compared to the mononuclear cell population that was used as the starting material. Additionally, the TRCs have both high viability and low residual levels of components used during their production.

The viability of the TRC's is at least 50%, 60%, 70%, 75%, 80%, 85%, 90%, 95% or more. Viable cells are cells which are of measurable cell viability. Viability can be measured by methods known in the art such as trypan blue exclusion. Cell viability can also be measured using a variety of cell viability assays including but not limited to measurement of metabolic activity (e.g.: MTT [3-(4, 5-dimethylthiazol-2-yl)-2, 5-diphenyltetrazolium bromide] assay, ATP [adenosine tri-phosphate] assay), survival and growth in tissue culture (e.g. proliferation assay), functional assay, metabolite incorporation (e.g. fluorescence-based assays), structural alteration, and membrane integrity (e.g. LDH (lactate dehydrogenase) assay). Each viability assay method is based on different definitions of cell viability. This enhanced viability makes the TRC population more effective in tissue repair, as well as enhances the shelf-life and cryopreservation potential of the final cell product.

By components used during production is meant, but not limited, to culture media components such as horse serum, fetal bovine serum and enzyme solutions for cell harvest. Enzyme solutions include trypsins (animal-derived, microbial-derived, or recombinant), various collagenases, alternative microbial-derived enzymes, dissociation agents, general proteases, or mixtures of these. Removal of these components provides safe administration of TRC to a subject in need thereof.

Preferably, the TRC compositions of the invention contain less than 10, 5, 4, 3, 2, 1 μg/ml bovine serum albumin; less than 5, 4, 3, 2, 1, 0.9, 0.8, 0.7, 0.6, 0.5 μg/ml harvest enzymes (as determined by enzymatic activity) and are substantially free of mycoplasm, endotoxin and microbial (e.g., aerobic, anaerobic and fungi) contamination.

By substantially free of endotoxin is meant that there is less endotoxin per dose of TRCs than that is allowed by the FDA for a biologic, which is a total endotoxin of 5 EU/kg body weight per day, which for an average 70 kg person is 350 EU per total dose of TRCs.

By substantially free for mycoplasma and microbial contamination is meant as negative readings for the generally accepted tests known to those skilled in the art. For example, mycoplasm contamination is determined by subculturing a TRC product sample in broth medium and distributed over agar plates on day 1, 3, 7, and 14 at 37° C. with appropriate positive and negative controls. The product sample appearance is compared microscopically, at 100×, to that of the positive and negative control. Additionally, inoculation of an indicator cell culture is incubated for 3 and 5 days and examined at 600× for the presence of mycoplasmas by epifluorescence microscopy using a DNA-binding fluorochrome. The product is considered satisfactory if the agar and/or the broth media procedure and the indicator cell culture procedure show no evidence of mycoplasma contamination.

The sterility test to establish that the product is free of microbial contamination is based on the U.S. Pharmacopedia Direct Transfer Method. This procedure requires that a pre-harvest medium effluent and a pre-concentrated sample be inoculated into a tube containing tryptic soy broth media and fluid thioglycollate media. These tubes are observed periodically for a cloudy appearance (turbidity) for a 14-day incubation. A cloudy appearance on any day in either medium indicates contamination, while a clear appearance (no growth) testing indicates substantially free of contamination.

The ability of cells within TRCs to form clonogenic colonies compared to BMMNCs was determined. Both hematopoietic (CFU-GM) and mesenchymal (CFU-F) colonies were monitored (Table 1). As shown in Table 1, while CFU-F were increased 280-fold, CFU-GM were slightly decreased by culturing.

TABLE 1

| | BMMNC Input | TRC Output | |
|---|---|---|---|
| | (E−06) | (E−06) | Fold Exp |
| CFU-GM | 1.7 | 1.1 ± 0.2 | 0.7 ± 0.1 |
| CFU-F | 0.03 | 6.7 ± 1.3 | 280 ± 67 |

Results are the average ± SEM from 8 clinical-scale experiments.

The cells of the TRC composition have been characterized by cell surface marker expression. Table 2 shows the typical phenotype measured by flow cytometry for starting BMMNCs and TRCs. (See, Table 2). These phenotypic and functional differences highly differentiate TRCs from the mononuclear cell starting compositions.

TABLE 2

| | | BMMNC Input | | TRC Output | | |
|---|---|---|---|---|---|---|
| | | | Total | | Total | |
| | | | (in | | (in | Fold |
| Lineage | Marker | % | millions) | % | millions) | Expansion |
| M | CD105/166 | 0.03 | 0.1 | 12 | 16 | 373 |
| H | CD14auto⁺ | 0.2 | 0.5 | 26 | 36 | 81 |
| M | CD90 | 0.4 | 0.9 | 22 | 28 | 39 |
| H (E) | CXCR4/ | 0.7 | 1.9 | 12 | 9.9 | 21 |
| | VEGFR1 | | | | | |
| E | CD144/146 | 0.5 | 1.3 | 2.7 | 3.2 | 6.3 |
| E | VEGFR1 | 7.6 | 22 | 26 | 38 | 2.3 |
| E | VEGFR2 | 12 | 37 | 25 | 37 | 1.3 |
| H | CD14auto⁻ | 11 | 31 | 14 | 17 | 0.9 |
| H | CD11b | 59 | 162 | 64 | 83 | 0.5 |
| H | CD45 | 97 | 269 | 80 | 104 | 0.4 |
| H | CD3 | 24 | 67 | 8.6 | 11 | 0.2 |

M = mesenchymal lineage,
H = hematopoietic lineage,
E = endothelial lineage.
Results are the average of 4 clinical-scale experiments.

Markers for hematopoietic, mesenchymal, and endothelial lineages were examined. Average results from 4 experiments comparing starting BMMNC and TRC product are shown in Figures. Most hematopoietic lineage cells, including CD11b myeloid, CD14auto-monocytes, CD34 progenitor, and CD3 lymphoid, are decreased slightly, while CD14auto⁺ macrophages, are expanded 81-fold. The mesenchymal cells, defined by CD90⁺ and CD105^{|}/CD166^{|}/CD45⁻/CD14⁻ have expansions up to 373-fold. Cells that may be involved in vascularization, including mature vascular endothelial cells (CD144/CD146) and CXCR4NEGFR1⁺ supportive cells have expansions between 6- to 21-fold.

Although most hematopoietic lineage cells do not expand in these cultures, the final product still contains close to 80% CD45⁺ hematopoietic cells and approximately 20% CD90⁺ mesenchymal cells.

The TRC are highly enriched for CD90⁺ cells compared to the mononuclear cell population from which they are derived. The cells in the TRC composition are at least 5%, 10%, 25%, 50%, 75%, or more CD90⁺. The remaining cells in the TRC composition are CD45⁺ Alternatively, the remaining cells in the TRC composition are CD45⁺, CD31⁺, CD14⁺ and/or auto Preferably, the cells in the TRC composition are about 5-75% viable CD90⁺. In various aspects, at least 5%, 10%, 15% , 20%, 25%, 30%, 40%, 50%, 60% or more of the CD90⁺ are also CD15⁺ (see Table 3). In addition, the CD90⁺ are also CD105⁺.

TABLE 3

| | TRC | TRC |
|---|---|---|
| | Run 1 | Run 2 |
| % CD90⁺ | 29.89 | 18.08 |
| % CD90⁺ CD15− | 10.87 | 3.18 |
| % CD90⁺ CD15⁺ | 19.02 | 14.90 |
| % CD15⁺ of the CD90s | 63.6 | 82.4 |

In contrast, the CD90⁻ population in bone marrow mononuclear cells (BMMNC) is typically less than 1% with the resultant CD45⁺ cells making up greater than 99% of the nucleated cells in BMMNCs Thus, there is a significant reduction of many of the mature hematopoietic cells in the TRC composition compared to the starting mononuclear cell population (see Table 2).

This unique combination of hematopoietic, mesenchymal and endothelial stems cells are not only distinct from mononuclear cells but also other cell compositions currently being used in cell therapy. Table 4 demonstrates the cell surface marker profile of TRC compared to mesenchymal stem cells and adipose derived stem cells. (Deans R J, Moseley A B. 2000. Exp. Hematol. 28: 875-884; Devine S M. 2002. J Cell Biochem Supp 38: 73-79; Katz A J, et al. 2005. Stem Cells. 23:412-423; Gronthos S, et al. 2001. J Cell Physiol 189:54-63; Zuk P A, et al. 2002. Mol Biol Cell. 13: 4279-95.)

For example, mesenchymal stem cells (MSCs) are highly purified for CD90⁺ (greater than 95% CD90⁻), with very low percentage CD45⁻ (if any). Adipose-derived stem cells are more variable but also typically have greater than 95% CD90^{|}, with almost no CD45^{|} blood cells as part of the composition. There are also Multi-Potent Adult Progenitor Cells (MAPCs), which are cultured from BMMNCs and result in a pure CD90 population different from MSCs that co-expresses CD49c. Other stem cells being used are highly purified cell types including CD34⁺ cells, AC133⁺ cells, and CD34⁺lin⁻ cells, which by nature have little to no CD90⁺ cells as part of the composition and thus are substantially different from TRCs.

Cell marker analysis have also demonstrated that the TRCs isolated according to the methods of the invention have higher percentages of CD14⁺, auto⁺, CD34⁺ and VEGFR⁺ cells.

TABLE 4

| | | | | Adipose- |
|---|---|---|---|---|
| | | | Mesenchymal | Derived Stem |
| CD Locus | Common Name | TRC | stem cells | Cells |
| CD 34 | − | + | − | ± |
| CD13 | gp150 | + | Na | + |
| CD15 | LewisX, SSEA-1 | + | − | − |
| CD11b | Mac-1 | + | − | ± |
| CD14 | LPS receptor | + | − | − |
| CD235a | glycophorin A | + | Na | Na |
| CD45 | Leukocyte common | + | − | − |
| | antigen | | | |
| CD90 | Thy1 | + | + | + |
| CD105 | Endoglin | + | + | + |
| CD166 | ALCAM | + | + | + |
| CD44 | Hyaluronate | + | + | + |
| | receptor | | | |
| CD133 | AC133 | + | − | ± |
| — | vWF | + | Na | Na |
| CD144 | VE-Cadherin | + | − | + |
| CD146 | MUC18 | + | + | Na |
| CD309 | VEGFR2, KDR | + | Na | Na |

Each of the cell types present in a TRC population has varying immunomodulatory properties. Monocytes/macrophages (CD45⁺, CD14⁺) inhibit T cell activation, as well as showing indoleamine 2,3-dioxygenase (IDO) expression by the macrophages. (Munn D. H. and Mellor A. L., Curr Pharm Des., 9:257-264 (2003); Munn D. H., et al. J Exp Med., 189:1363-1372 (1999); Mellor A. L. and Munn D. H., J. Immunol., 170:5809-5813 (2003); Munn D H., et al., J. Immunol., 156:523-532 (1996)). Monocytes and macrophages regulate inflammation and tissue repair. (Duffield J. S., Clin Sci (Lond), 104:27-38 (2003); Gordon, S.; Nat. Rev. Immunol., 3:23-35 (2003); Mosser, D. M., J. Leukoc. Biol., 73:209-212 (2003); Philippidis P., et al., Circ. Res., 94:119-126 (2004). These cells also induce tolerance and transplant immunosuppression. (Fandrich F et al. Hum. Immunol., 63:805-812 (2002)). Regulatory T-cells (CD45⁺ CD4⁺ CD25⁺) regulate innate inflammatory response after injury. (Murphy T. J., et al., J. Immunol., 174:2957-2963 (2005)). The T-cells are also responsible for maintenance of self tolerance and prevention and suppression of autoimmune disease. (Sakaguchi S. et al., Immunol. Rev., 182:18-32 (2001); Tang Q., et al., J. Exp. Med., 199:1455-1465 (2004)) The T-cells also induce and maintain transplant tolerance (Kingsley C. I., et al. J. Immunol., 168:1080-1086 (2002); Graca L., et al., J. Immunol., 168:5558-5565 (2002)) and inhibit graft versus host disease (Ermann J., et al., Blood, 105:2220-2226 (2005); Hoffmann P., et al., Curr. Top. Microbiol. Immunol., 293:265-285 (2005); Taylor P. A., et al., Blood, 104:3804-3812 (2004). Mesenchymal stem cells (CD45⁻ CD90⁺ CD105⁺) express IDO and inhibit T-cell activation (Meisel R., et al., Blood, 103:4619-4621 (2004); Krampera M., et al., Stem Cells, (2005)) as well as induce anti-inflammatory activity (Aggarwal S. and Pittenger M. F., Blood, 105:1815-1822 (2005)).

TRCs also show increased expression of programmed death ligand 1 (PDL1). Increased expression of PDL1 is associated with production of the anti-inflammatory cytokine IL-10. PDL1 expression is associated with a non-inflammatory state. TRCs have increased PDL1 expression in response to inflammatory induction, showing another aspect of the anti-inflammatory qualities of TRCs.

TRCs, in contrast to BMMNCs also produce at least five distinct cytokines and one regulatory enzyme with potent activity both for wound repair and controlled down-regulation of inflammation Specifically, TRCs produce 1) Interleukin-6 (IL-6), 2) Interleukin-10 (IL-10), 3) vascular endothelial growth factor (VEGF), 4) monocyte chemoattractant protein-1 (MCP-1) and, 5) interleukin-1 receptor antagonist (IL-1ra). The characteristics of these five cytokines are summarized in Table 5, below.

TABLE 5
Characteristics of TRC Expressed Cytokines.

| CYTOKINE | CHARACTERISTIC |
|---|---|
| IL-1 ra | Decoy receptor for IL-1 down-regulates inflammation. |
| | IL-1 ra and IL-10 are characteristically produced by |
| | alternatively activated macrophages |
| IL-6 | Interleukin-6 (IL-6) is a pleiotropic cytokine with a wide |
| | range of biological activities. This cytokine regulates |
| | polarization of naive CD4⁺ T-cells toward the Th2 |
| | phenotype, further promotes Th2 differentiation by |
| | up-regulating NFAT1 expression and inhibits |
| | proinflammatory Thl differentiation by inducing |
| | suppressor of cytokine signaling SOCS1. |
| IL-10 | Produced by cell types mediating anti-inflammatory |
| | activities, Th2 type immunity, immunosuppression and |
| | tissue repair. IL-10 and IL-1ra are characteristically |
| | produced by alternatively activated macrophages. |
| | IL-10 also is involved in the induction of regulatory |
| | T-cells. In addition, regulatory T-cells secrete high |
| | levels of IL-10. |
| MCP-1 | MCP-1 inhibits the adoptive transfer of autoimmune disease |
| | in animal models and drives TH2 differentiation |
| | indicating an anti inflammatory property particularly |
| | when balanced a against MIP-1α. |
| VEGF | Angiogenic cytokine with simultaneous immunosuppressive |
| | properties acting at the level of the antigen |
| | presenting cell. |

Additional characteristics of TRCs include a failure to spontaneously produce, or very low-level production of certain pivotal mediators known to activate the Th1 inflammatory pathway including interleukin-alpha (IL-1α), interleukin-beta (IL-1β) interferon-gamma (IFN-γ) and most notably interleukin-12 (IL-12). Importantly, the TRCs neither produce these latter Th1-type cytokines spontaneously during medium replacement or perfusion cultures nor after intentional induction with known inflammatory stimuli such as bacterial lipopolysaccharide (LPS). TRCs produced low levels of IFN-γ only after T-cell triggering by anti-CD3 mAb. Finally, the TRCs produced by the current methods produce more of the anti-inflammatory cytokines IL-6 and IL-10 as well as less of the inflammatory cytokine IL-12.

Moreover, TRCs are inducible for expression of a key immune regulatory enzyme designated indoleamine-2,-3 dioxygenase (IDO). The TRCs according to the present invention express higher levels of IDO upon induction with interferon-y. IDO has been demonstrated to down-regulate both nascent and ongoing inflammatory responses in animal models and humans (Meisel R., et al., Blood, 103:4619-4621 (2004); Munn D. H., et al., J. Immunol., 156:523-532 (1996); Munn D. H., et al. J. Exp. Med. 189:1363-1372 (1999); Munn D. H. and Mellor A. L., Curr. Pharm. Des., 9:257-264 (2003); Mellor A. L. and Munn D. H., J. Immunol., 170:5809-5813 (2003)).

As discussed above, TRCs are highly enriched for a population of cells that co-express CD90 and CD15.

CD90 is present on stem and progenitor cells that can differentiate into multiple lineages. These cells are a heterogeneous population of cells that are at different states of differentiation. Cell markers have been identified on stem cells of embryonic or fetal origin that define the differentiation state of the cell. One of these markers, SSEA-1, also referred to as CD15, is found on mouse embryonic stem cells, but is not expressed on human embryonic stem cells. It has however been detected in neural stem cells in both mices and human. CD15 is also not expressed on purified mesenchymal stem cells derived from human bone marrow or adipose tissue (see Table 6). Thus, the cell population in TRCs that co-expresses both CD90 and CD15 is a unique cell population and may define a stem-like state of the CD90 adult-derived cells.

Accordingly, in another aspect of the invention the cell population expressing both CD90 and CD15 may be further enriched. By further enriched is meant that the cell composition contains 5%, 10%, 25%, 50%, 75%, 80%, 85%, 90%, 95%, 96%, 97%, 98% 99% or 100% CD90⁺ CD15⁺ cells. TRCs can be further enriched for CD90⁺ CD15⁺ cells by methods known in the art such as positive or negative selection using antibodies direct to cell surface markers. The TRCs that have been further enriched for CD90⁺ CD15⁺ cells are particularly useful in cardiac repair and regeneration.

TABLE 6

| Cell Phenotype | TRC | MSC P0 |
|---|---|---|
| % CD90⁺ | 23.99 | 98.64 |
| % CD15⁺ | 39.89 | 0.76 |
| % CD15⁺/CD90⁺ | 19.54 | 0.22 |
| N | 2 | 4 |

The CFU-F and osteogenic potential of CD90⁺ CD15⁺ was assessed. When CD90⁺ cells are removed, all CFU-F and in vitro osteogenic potential is depleted. Suprisingly, although the overall frequency of CD90 and CFU-F are higher in MSC cultures (where CD90 do not express CD15), the relative number of CFU-F per CD90 cells is dramtically higher in TRC. This demonstrates that the CD90 cells are much more potent in TRCs when grown as purified cell populations.

### Methods of Production of TRCs

TRCs are isolated from any mammalian tissue that contains bone marrow mononuclear cells (BMMNC). Suitable sources for BMMNC is peripheral blood, bone marrow, umbilical cord blood or fetal liver. Blood is often used because this tissue is easily obtained. Mammals include for example, a human, a primate, a mouse, a rat, a dog, a cat, a cow, a horse or a pig.

The culture method for generating TRCs begins with the enrichment of BMMNC from the starting material (e.g., tissue) by removing red blood cells and some of the polynucleated cells using a conventional cell fractionation method. For example, cells are fractionated by using a FICOLL® density gradient separation. The volume of starting material needed for culture is typically small, for example, 40 to 50 mL, to provide a sufficient quantity of cells to initiate culture. However, any volume of starting material may be used.

Nucleated cell concentration is then assessed using an automated cell counter, and the enriched fraction of the starting material is inoculated into a biochamber (cell culture container). The number of cells inoculated into the biochamber depends on its volume. TRC cultures which may be used in accordance with the invention are performed at cell densities of from 10⁴ to 10⁹ cells per ml of culture. When a Aastrom Replicell Biochamber is used, 2-3×10⁸ total cells are inoculated into a volume of approximately 280 mL.

Prior to inoculation, a biochamber is primed with culture medium. Illustratively, the medium used in accordance with the invention comprises three basic components. The first component is a media component comprised of IMDM, MEM, DMEM, RPMI 1640, Alpha Medium or McCoy's Medium, or an equivalent known culture medium component. The second is a serum component which comprises at least horse serum or human serum and may optionally further comprise fetal calf serum, newborn calf serum, and/or calf serum. Optionally, serum free culture mediums known in the art may be used. The third component is a corticosteroid, such as hydrocortisone, cortisone, dexamethasone, solumedrol, or a combination of these, preferably hydrocortisone. The culture medium further comprises B7H3 polypeptides, VSIG4 polypeptides or a combination of both. When the Aastrom Replicell Biochamber is used, the culture medium consists of IMDM, about 10% fetal bovine serum, about 10% horse serum, about 5 μM hydrocortisone, and 4 mM L-Glutamine. The cells and media are then passed through the biochamber at a controlled ramped perfusion schedule during culture process. The cells are cultures for 2, 4, 6, 8, 10, 12, 14, 16 or more days. Preferably, the cells are cultured for less than 12 days. Not to be bound by theory, but it is thought that the addition of B7H3 polypeptides, VSIG4 polypeptides or both will allow for the rapid expansion of TRCs, in particular the CD45⁺, CD31⁺, CD14⁺, and auto⁺ cell population. This rapid expansion will greatly reduce culturing time which is a particular advantage when manufacturing cell suitable for transplantation into humans.

For example, when used with the Aastrom Replicell System Cell Cassette, the cultures are maintained at 37° C. with 5% CO₂ and 20% O₂.

These cultures are typically carried out at a pH which is roughly physiologic, i.e. 6.9 to 7.6. The medium is kept at an oxygen concentration that corresponds to an oxygen-containing atmosphere which contains from 1 to 20 vol. percent oxygen, preferably 3 to 12 vol. percent oxygen. The preferred range of O₂ concentration refers to the concentration of O₂ near the cells, not necessarily at the point of O₂ introduction which may be at the medium surface or through a membrane.

Standard culture schedules call for medium and serum to be exchanged weekly, either as a single exchange performed weekly or a one-half medium and serum exchange performed twice weekly. Preferably, the nutrient medium of the culture is replaced, preferably perfused, either continuously or periodically, at a rate of about 1 ml per ml of culture per about 24 to about 48 hour period, for cells cultured at a density of from 2×10⁶ to 1×10⁷ cells per ml. For cell densities of from 1×10⁴ to 2×10⁶ cells per ml the same medium exchange rate may be used. Thus, for cell densities of about 10⁷ cells per ml, the present medium replacement rate may be expressed as 1 ml of medium per 10⁷ cells per about 24 to about 48 hour period. For cell densities higher than 10⁷ cells per ml, the medium exchange rate may be increased proportionality to achieve a constant medium and serum flux per cell per unit time

A method for culturing bone marrow cells is described in Lundell, et al., “Clinical Scale Expansion of Cryopreserved Small Volume Whole Bone Marrow Aspirates Produces Sufficient Cells for Clinical Use,” J. Hematotherapy (1999) 8:115-127 (which is incorporated herein by reference). Bone marrow (BM) aspirates are diluted in isotonic buffered saline (Diluent 2, Stephens Scientific, Riverdale, N.J.), and nucleated cells are counted using a Coulter ZM cell counter (Coulter Electronics, Hialeah, Fla.). Erythrocytes (non-nucleated) are lysed using a Manual Lyse (Stephens Scientific), and mononuclear cells (MNC) are separated by density gradient centrifugation (Ficoll-Paque® Plus, Pharmacia Biotech, Uppsala, Sweden) (specific gravity 1.077) at 300 g for 20 min at 25° C. BMMNC are washed twice with long-term BM culture medium (LTBMC) which is Iscove's modified Dulbecco's medium (IMDM) supplemented with 4 mM L-glutamine 9GIBCO BRL, Grand Island, N.Y.), 10% fetal bovine serum (FBS), (Bio-Whittaker, Walkersville, Md.), 10% horse serum (GIBCO BRL), 20 μg/ml vancomycin (Vancocin® HCl, Lilly, Indianapolis, Ind.), 5 μg/ml gentamicin (Fujisawa USA, Inc., Deerfield, Ill.), and 5 μM hydrocortisone (Solu-Cortef®, Upjohn, Kalamazoo, Mich) before culture.

### Cell Storage

After culturing, the cells are harvested, for example using trypsin, and washed to remove the growth medium. The cells are resuspended in a pharmaceutical grade electrolyte solution, for example Isolyte (B. Braun Medical Inc., Bethlehem, Pa.) supplemented with serum albumin.

Alternatively, the cells are washed in the biochamber prior to harvest using the wash harvest procedure described below. Optionally after harvest the cells are concentrated and cryopreserved in a biocompatible container, such as 250 ml cryocyte freezing containers (Baxter Healthcare Corporation, Irvine, Calif.) using a cryoprotectant stock solution containing 10% DMSO (Cryoserv, Research Industries, Salt Lake City, Utah), 10% HSA (Michigan Department of Public Health, Lansing, Mich), and 200 μg/ml recombinant human DNAse (Pulmozyme®, Genentech, Inc., South San Francisco, Calif.) to inhibit cell clumping during thawing. The cryocyte freezing container is transferred to a precooled cassette and cryopreserved with rate-controlled freezing (Model 1010, Forma Scientific, Marietta, Ohio). Frozen cells are immediately transferred to a liquid nitrogen freezer (CMS-86, Forma Scientific) and stored in the liquid phase. Preferred volumes for the concentrated cultures range from about 5 mL to about 15 ml. More preferably, the cells are concentrated to a volume of 7.5 mL.

### Post-Culture

When harvested from the biochamber the cells reside in a solution that consists of various dissolved components that were required to support the culture of the cells as well as dissolved components that were produced by the cells during the culture. Many of these components are unsafe or otherwise unsuitable for patient administration. To create cells ready for therapeutic use in humans it is therefore required to separate the dissolved components from the cells by replacing the culture solution with a new solution that has a desired composition, such as a pharmaceutical-grade, injectable, electrolyte solution suitable for storage and human administration of the cells in a cell therapy application.

A significant problem associated with many separation processes is cellular damage caused by mechanical forces applied during these processes, exhibited, for instance, by a reduction in viability and biological function of the cells and an increase in free cellular DNA and debris. Additionally, significant loss of cells can occur due to the inability to both transfer all the cells into the separation apparatus as well as extract all the cells from the apparatus.

Separation strategies are commonly based on the use of either centrifugation or filtration. An example of centrifugal separation is the COBE 2991 Cell Processor (COBE BCT) and an example of a filtration separation is the CYTOMATE® Cell Washer (Baxter Corp) (Table 7). Both are commercially available state-of-the-art automated separation devices that can be used to separate (wash) dissolved culture components from harvested cells. As can be seen in Table 7, these devices result in a significant drop in cell viability, a reduction in the total quantity of cells, and a shift in cell profile due to the preferential loss of the large and fragile CD14⁺ auto⁺ subpopulation of TRCs.

TABLE 7
Performance of 2 different cell separation devices, 3 different studies.

| | COBE 2991 Cell | CYTOMATE ® Cell | CYTOMATE ® Cell |
|---|---|---|---|
| | Processor (n = 3) | Washer (n = 8) | Washer (n = 26) |
| Operating principal | Centrifugation | Filtration | Filtration |
| Study Reference | Aastrom internal | Aastrom new wash | US Fracture Clinical |
| | protocol report | process development, | Trial, BB-IND #10486 |
| | #PABI0043 | report MF#0384 | |
| Average pre-separation | 93% | 93% | 95% |
| cell viability | | | |
| Average post- | 83% | 71% | 81% |
| separation cell viability | | | |
| Average reduction in | 18% | 69% | Not available |
| CD14⁺Auto⁺ frequency | | | |
| Average cell recovery | 73% | 74% | Not available |

These limitations in the art create difficulties in implementing manufacturing and production processes for creating cell populations suitable for human use. It is desirable for the separation process to minimize damage to the cells and thereby result in a cell solution that is depleted of unwanted dissolved components while retaining high viability and biological function with minimal loss of cells. Additionally, it is important to minimize the risk of introducing microbial contaminants that will result in an unsafe final product. Less manipulation and transfer of the cells will inherently reduce this risk.

The invention described in this disclosure overcomes all of these limitations in the current art by implementing a separation process to wash the cells that minimizes exposure of the cells to mechanical forces and minimizes entrapment of cells that cannot be recovered. As a result, damage to cells (e.g. reduced viability or function), loss of cells, and shift in cell profile are all minimized while still effectively separating unwanted dissolved culture components. In a preferred implementation, the separation is performed within the same device that the cells are cultured in which eliminates the added risk of contamination by transfer and separation using another apparatus. The wash process according to the invention is described below.

### Wash Harvest

As opposed to conventional culture processes where cells are removed (harvested) from the biochamber followed by transfer to another apparatus to separate (wash) the cells from culture materials, the wash-harvest technique reverses the order and provides a unique means to complete all separation (wash) steps prior to harvest of the cells from the biochamber.

To separate the culture materials from the cells, a new liquid of desired composition (or gas) may be introduced, preferably at the center of the biochamber and preferably at a predetermined, controlled flow rate. This results in the liquid being displaced and expelled along the perimeter of the biochamber, for example, through apertures 48, which may be collected in the waste bag 76.

In some embodiments of the invention, the diameter of the liquid space in the biochamber is about 33 cm, the height of the liquid space is about 0.33 cm and the flow rates of adding rinsing and/or harvesting fluids to the biochamber is about 0.03 to 1.0 volume exchanges (VE) per minute and preferably 0.50 to about 0.75 VE per minute. This substantially corresponds to about 8.4 to about 280 mL/min and preferably 140 to about 210 ml/min. The flow rates and velocities, according to some embodiments, aid in insuring that a majority of the cultured cells are retained in the biochamber and not lost into the waste bag and that an excessively long time period is not required to complete the process. Generally, the quantity of cells in the chamber may range from 10⁴ to 10⁸ cell/mL. For TRCs, the quantity may range from 10⁵ to 10⁶ cells/mL, corresponding to 30 to 300 million total cells for the biochamber dimensions above. Of course, one of skill in the art will understand that cell quantity changes upon a change in the biochamber dimensions

According to some embodiments, in harvesting the cultured cells from the biochamber, the following process may be followed, and is broadly outlined in Table 3, below. The solutions introduced into the biochamber are added into the center of the biochamber. The waste media bag 76 may collect corresponding fluid displaced after each step where a fluid or gas is introduced into the biochamber. Accordingly, after cells are cultured, the biochamber is filled with conditioned culture medium (e.g., IMDM, 10% FBS, 10% Horse Serum, metabolytes secreted by the cells during culture) and includes between about 30 to about 300 million cells. A 0.9% NaCl solution (“rinse solution”) may then be introduced into the biochamber at about 140 to 210 mL per minute until about 1.5 to about 2.0 liters of total volume has been expelled from the biochamber (Step 1).

While a single volume exchange for introduction of a new or different liquid within the biochamber significantly reduces the previous liquid within the biochamber, some amount of the previous liquid will remain. Accordingly, additional volume exchanges of the new/different liquid will significantly deplete the previous liquid.

Optionally, when the cells of interest are adherent cells, such as TRCs, the rinse solution is replaced by harvest solution. A harvest solution is typically an enzyme solution that allows for the detachment of cells adhered to the culture surface. Harvest solutions include for example 0.4% Trypsin/EDTA in 0.9% NaCl that may be introduced into the biochamber at about 140 to 210 mL per minute until about 400 to about 550 ml of total volume has been delivered (Step 2). Thereafter, a predetermined period of time elapses (e.g., 13-17 minutes) to allow enzymatic detachment of cells adhered to the culture surface of the biochamber (Step 3).

Isolyte (B Braun) supplemented with 0.5% HSA may be introduced at about 140 to 210 mL per minute until about 2 to about 3 liters of total volume has been delivered, to displace the enzyme solution (Step 4).

At this point, separation of unwanted solutions (culture medium, enzyme solution) from the cells is substantially complete.

To reduce the volume collected, some of the Isolyte solution is preferably displaced using a gas (e.g., air) which is introduced into the biochamber at a disclosed flow rate (Step 5). This may be used to displace approximately 200 to 250 cc of the present volume of the biochamber.

The biochamber may then be agitated to bring the settled cells into solution (Step 6). This cell suspension may then be drained into the cell harvest bag 70 (or other container) (Step 7). An additional amount of the second solution may be added to the biochamber and a second agitation may occur in order to rinse out any other residual cells (Steps 8 & 9). This final rinse may then be added to the harvest bag 70 (Step 10).

TABLE 8
Wash-harvest Protocol

| Step Number & Name | | Description |
|---|---|---|
| 1 | Rinse out culture media | Use Sodium Chloride to displace the culture medium into |
| | | the waste container. |
| 2 | Add Trypsin solution | Replace Sodium Chloride in culture chamber with the |
| | | Trypsin solution. |
| 3 | Trypsin incubation | Static 15 minute incubation in Trypsin solution. |
| 4 | Rinse out Trypsin solution/Transfer | Add Isolyte with 0.5% HSA to displace the Trypsin |
| | in Pharmaceutically Acceptable | solution into the waste container. |
| | Carrier | |
| 5 | Concentration/Volume reduction | Displace some of the Isolyte solution with air to reduce |
| | | the final volume (concentration step) |
| 6 | Agitate Biochamber | Rocking motion to dislodge and suspend cells into Isolyte |
| | | solution for collection |
| 7 | Drain into Collection Container | Drain Cells in Isolyte solution into cell collection bag. |
| 8 | Add rinse solution to Biochamber | Add more Isolyte to rinse out residual cells. |
| 9 | Agitate Biochamber | Rocking motion to dislodge and suspend cells into Isolyte |
| | | solution for collection |
| 10 | Drain into Collection Container | Drain the final rinse into the cell collection bag. |

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated mesenchymal stromal cell composition wherein the mesenchymal stromal cells express B7 homolog 3 (B7-H3).

2. An isolated cell composition comprising mesenchymal stromal cells that express B7 homolog 3 (B7-H3).

3. The composition of claim 2, wherein the mesenchymal stromal cells are non-proliferative after less than 5 passages in culture.

4. The composition claim 2, wherein the mesenchymal stromal cells have been immortalized.

5. The composition of claim 2, wherein at least 90% of the cells express CD90.

6. The composition of claim 2, wherein at least 80% of the cells express CD90.

7. The composition of claim 2, wherein at least 70% of the cells express CD90.

8. The cell composition of claim 2, wherein the composition comprises less than 25% viable CD45⁺ cells.

9. The composition of claim 2, wherein the mesenchymal stromal cells are derived from hematopoietic cells.

10. The composition of claim 2, wherein the mesenchymal stromal cells are adherent in culture.

11. The composition of claim 2, wherein the mesenchymal stromal cells do not differentiate in culture.

12. The composition of claim 2, wherein said composition inhibits T-cell activation.

13. The composition of claim 2, wherein said composition promotes the expansion of Th2/CD8 lymphocytes.

14. The composition of claim 2, wherein the mesenchymal stromal cells are CD90⁺ and CD105⁺.

15. The composition of claim 14, wherein the mesenchymal stromal cells are CD146⁺ and CD73⁺.

16. The composition of claim 2, wherein the mesenchymal stromal cells are derived from mononuclear cells.

17. The composition of claim 16, wherein the mononuclear cells are derived from mobilized peripheral blood, bone marrow, umbilical cord blood or fetal liver.

18. A mesenchymal stromal cell that has been genetically engineered to stably express B7 homolog 3 (B7-H3) on the surface of the cell.

19. The composition of claim 2, wherein the composition contains
a) less than 2 μg/ml of bovine serum albumin;
b) less than 1 μg/ml of a enzymatically active harvest reagent; and
c) substantially free of mycoplasm, endotoxin, and microbial contamination.

20. A method of tissue regeneration or repair comprising administering to a patient in need thereof the composition of claim 2.

21. The method of claim 20, wherein said tissue is selected from the group consisting of cardiac tissue, bone tissue, neuronal tissue, skin tissue, lung tissue, salivary gland tissue, liver tissue, and pancreatic tissue.

22. A method of producing a cell composition comprising a mixed population of cells of hematopoietic, mesenchymal and endothelial lineage, wherein the cell composition is characterized as containing 5-75% viable CD90⁺ cells with the remaining cells in said composition being CD45⁺, CD31⁺, CD14⁺, and auto⁺, comprising culturing mononuclear cells in the presence of
i) B7-H3 polypeptide,
ii) a V-set and Ig domain-containing 4 (VSIG4) polypeptide, or
iii) both i) and ii).

23. A method of producing a substantially pure population of CD14+ autofluorescent macrophages comprising culturing mononuclear cells in the presence of
i) a B7-H3 polypeptide,
ii) a V-set and Ig domain-containing 4 (VSIG4) polypeptide, or
iii) both i) and ii);
and isolating said CD14⁺ autofluorescent macrophages from said culture.

24. A method of producing a substantially pure population of CD14⁺ autofluorescent macrophages comprising culturing mononuclear cells in the presence of the composition of claim 2 and isolating said CD14⁺ autofluorescent macrophages from said culture.

25. The method of claim 23, wherein the macrophages express at least one of the following markers: CD45, CD163 or CD206.

26. The method of claim 22, wherein the culturing is performed by:
providing a biochamber for culturing the mononuclear cells;
providing a culture media for culturing the mononuclear cells within the biochamber;
inoculating the biochamber with the mononuclear cells; and
culturing the mononuclear cells.

27. The method of claim 26, further comprising:
upon a predetermined time period of culture, displacing the culture media from the biochamber with a biocompatible first rinse solution;
replacing the first rinse solution with a cell harvest enzyme solution;
incubating the contents of the biochamber for a predetermined period of time, wherein during incubation, the enzyme at least dissociates the cells i) from each other, ii) from the biochamber surface, or iii) from each other and from the biochamber surface;
displacing the enzyme solution with a second rinse solution, wherein upon the enzyme being displaced, the chamber is substantially filled with the second rinse solution;
displacing a portion of the second rinse solution with a gas to obtain a predetermined reduced liquid volume in the chamber;
agitating the chamber to bring settled cells into suspension; and
draining the solution with suspended cells into a cell collection container.

28. The method of claim 23, wherein the culturing is performed by:
providing a biochamber for culturing the mononuclear cells;
providing a culture media for culturing the mononuclear cells within the biochamber;
inoculating the biochamber with the mononuclear cells; and
culturing the mononuclear cells.

29. The method of claim 28, further comprising:
upon a predetermined time period of culture, displacing the culture media from the biochamber with a biocompatible first rinse solution;
replacing the first rinse solution with a cell harvest enzyme solution;
incubating the contents of the biochamber for a predetermined period of time, wherein during incubation, the enzyme at least dissociates the cells i) from each other, ii) from the biochamber surface, or iii) from each other and from the biochamber surface;
displacing the enzyme solution with a second rinse solution, wherein upon the enzyme being displaced, the chamber is substantially filled with the second rinse solution;
displacing a portion of the second rinse solution with a gas to obtain a predetermined reduced liquid volume in the chamber;
agitating the chamber to bring settled cells into suspension; and
draining the solution with suspended cells into a cell collection container.

30. The method of claim 24, wherein the culturing is performed by:
providing a biochamber for culturing the mononuclear cells;
providing a culture media for culturing the mononuclear cells within the biochamber;
inoculating the biochamber with the mononuclear cells; and
culturing the mononuclear cells.

31. The method of claim 30, further comprising:
upon a predetermined time period of culture, displacing the culture media from the biochamber with a biocompatible first rinse solution;
replacing the first rinse solution with a cell harvest enzyme solution;
incubating the contents of the biochamber for a predetermined period of time, wherein during incubation, the enzyme at least dissociates the cells i) from each other, ii) from the biochamber surface, or iii) from each other and from the biochamber surface;
displacing the enzyme solution with a second rinse solution, wherein upon the enzyme being displaced, the chamber is substantially filled with the second rinse solution;
displacing a portion of the second rinse solution with a gas to obtain a predetermined reduced liquid volume in the chamber;
agitating the chamber to bring settled cells into suspension; and
draining the solution with suspended cells into a cell collection container.

32. The method of claim 24, wherein the macrophages express at least one of the following markers: CD45, CD163 or CD206.
